(19) United States
(12) Patent Application Publication
(10) Pub. No.: US 2019/0256451 A1
(43) Pub. Date: Aug. 22, 2019

(54) DISCRIMINATION OF CIS- AND TRANS-1,3-DIAMINOCYCLOHEXANES

(71) Applicant: BASF SE, Ludwigshafen am Rhein (DE)

(72) Inventors: Thomas INGRAM, Ludwigshafen (DE); Martin ERNST, Ludwigshafen (DE); Alexander PANCHENKO, Ludwigshafen (DE)

(73) Assignee: BASF SE, Ludwigshafen am Rhein (DE)

(21) Appl. No.: 16/307,999

(22) PCT Filed: Jun. 8, 2017
(86) PCT No.: PCT/EP2017/063931

§ 371 Date: Dec. 7, 2018

(30) Foreign Application Priority Data
Jun. 10, 2016 (EP) 16173915.6

(51) Int. Cl. C07C 209/86; C07D 487/08

(52) U.S. Cl. CPC C07C 209/86; C07D 487/08

(57) ABSTRACT

In a process for discriminating cis- and trans-1,3-diaminocyclohexanes, a mixture of cis- and trans-1,3-diaminocyclohexanes is reacted with carbon dioxide or a reactive carbonic acid derivative and the urea of the cis-1,3-diaminocyclohexane, which can be separated off by means of a mechanical separation method, is obtained selectively. 4-Methylcyclohexane-1,3-diamine and/or 2-methylcyclohexane-1,3-diamine having a trans content of 99 mol % or more are obtainable in this way.

The present invention relates to a process for discriminating cis- and trans-1,3-diaminocyclohexanes, and in particular for discriminating and separating cis- and trans-4-methylcyclohexane-1,3-diamine or cis- and trans-2-methylcyclohexane-1,3-diamine.

1,3-Diaminocyclohexanes are, for example, obtainable by hydrogenation of 1,3-phenylenediamines. Such a process is described in U.S. Pat. No. 6,075,167. Here, a stereoisomeric mixture of cis- and trans-1,3-diaminocyclohexanes in different proportions is obtained. For particular applications, it is necessary to have the pure cis or trans stereoisomers. Since the physical properties of the stereoisomers are very similar, a separation, e.g. by fractional distillation, is very difficult. A complete separation is virtually impossible using conventional methods.

Green Chem., 2007, 9, 158-161 describes the Cs⁺-catalyzed reaction of CO₂ and amines. Green Chem., 2008, 10, 465-469 describes the synthesis catalyzed by ionic liquids of disubstituted ureas from amines and CO₂.

According to Phys. Chem. Chem. Phys., 2012, 14, 464-468, polyureas can be obtained from diamines and CO₂ in the absence of catalysts and solvents.

It is an object of the invention to provide a process for discriminating cis- and trans-1,3-diaminocyclohexanes, which process is simple, economical and efficient. A further object of the invention is to provide 4-methylcyclohexane-1,3-diamine, 2-methylcyclohexane-1,3-diamine or mixtures thereof, in which the amino groups are arranged virtually exclusively in the trans orientation relative to the cyclohexane ring plane.

The object is achieved by a process for discriminating cis- and trans-1,3-diaminocyclohexanes, wherein a mixture of cis- and trans-1,3-diaminocyclohexanes is reacted with carbon dioxide or a reactive carbonic acid derivative and the urea of cis-1,3-diaminocyclohexane is obtained selectively.

The indication of configuration cis or trans in cis- and trans-1,3-diaminocyclohexane relates to the relative arrangement of the amino groups relative to the cyclohexane ring plane. It can be seen that the number of stereoisomers is higher when further substituents are present in addition to the two amino groups on the cyclohexane ring. For the purposes of the present invention, these stereoisomers are divided into two groups, namely a group in which the amino groups are in cis positions relative to one another and a group in which the amino groups are in trans positions relative to one another.

In particular embodiments, the 1,3-diaminocyclohexane bears at least one C₁-C₆-alkyl group in the a position relative to at least one amino group. A preferred 1,3-diaminocyclohexane is 4-methylcyclohexane-1,3-diamine, 2-methylcyclohexane-1,3-diamine or a mixture thereof. The mixture particularly preferably comprises from 50 to 95% by weight of 4-methylcyclohexane-1,3-diamine and from 5 to 50% by weight of 2-methylcyclohexane-1,3-diamine, based on the total amount of 1,3-diaminocyclohexane.

The invention also provides 4-methylcyclohexane-1,3-diamine, 2-methylcyclohexane-1,3-diamine or a mixture thereof having a trans content of 99 mol % or more, based on the sum of 4-methylcyclohexane-1,3-diamine and 2-methylcyclohexane-1,3-diamine.

It has been found that in a mixture of cis- and trans-1,3-diaminocyclohexanes, the cis stereoisomer is selectively converted into the urea, namely 2,4-diazabicyclo[3.3.1]nonan-3-one, in the presence of carbon dioxide or a reactive carbonic acid derivative. The reaction of the trans stereoisomer with carbon dioxide or a reactive carbonic acid derivative stops at the stage of the carbamate; an intramolecular ring closure does not occur. During the course of the work-up, e.g. by stripping off of CO₂ and/or thermally, the carbamate of the trans stereoisomer can be redissociated into the free trans-1,3-diaminocyclohexane.

The urea reaction product of cis-4-methylcyclohexane-1,3-diamine is illustrated below:

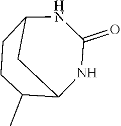

The urea derivative can easily be separated off from the trans-1,3-diaminocyclohexane, e.g. by precipitation, crystallization or distillation. In general, the urea reaction product is sparingly soluble in aqueous solutions and precipitates. The precipitation can be completed by cooling. The separation preferably comprises a mechanical separation method such as filtration, sedimentation and/or centrifugation, among which filtration is preferred.

The reactive carbonic acid derivative is, for example, selected from among urea, carbonic acid esters, e.g. dialkyl carbonates such as dimethyl carbonate, diethyl carbonate; alkylene carbonates such as ethylene carbonate, propylene carbonate; and phosgene.

The conditions in the reaction depend on the reactivity of the reactive carbonic acid derivative. In general, the reaction is carried out at elevated temperature, e.g. from 25 to 300° C., preferably from 25 to 200° C. The reaction can be carried out without dilution or in the presence of a solvent.

Carbon dioxide is the preferred reagent. The reaction with CO₂ can be carried out in a single stage under superatmospheric pressure, e.g. from 1.5 to 250 bar, preferably from 5 to 200 bar, particularly preferably from 10 to 60 bar, and at elevated temperature, e.g. from 25 to 300° C., preferably from 100 to 250° C. It can also be carried out in two stages, e.g. in a loading stage at low pressure and low temperature or high pressure and low temperature and a heating stage at higher temperature and variable pressure. Excess CO₂ can be vented between these stages and recirculated. The reaction time or residence time in the case of a process carried out continuously can be from 0.1 to 24 hours per stage.

For the reaction with CO₂, the mixture of cis- and trans-1,3-diaminocyclohexanes is generally present as a solution, but does not necessarily have to be diluted with another substance. Suitable solvents are water, alcohol, ethers. The reaction with CO₂ is preferably carried out in aqueous solution. For this purpose, an aqueous solution of cis-and trans-1,3-diaminocyclohexanes loaded with carbon dioxide can be heated under autogenous pressure in a pressure vessel. The degree of loading with CO₂ (expressed as mol(CO₂) per mol(1,3-diaminocyclohexane)) is preferably at least 0.5, in particular at least 1.0.

The reaction can also be carried out continuously or semicontinuously. A reactor cascade comprising a presaturation reactor and a carboxylation reactor is suitable. In the presaturation reactor, a solution of cis- and trans-1,3-diaminocyclohexane is presaturated with carbon dioxide by introduction of carbon dioxide, preferably at a temperature of from 10 to 50° C., e.g. room temperature. The solution loaded with CO₂ is then reacted at elevated pressure and elevated temperature in the carboxylation reactor, with the cis stereoisomer reacting selectively to form the urea.

In a preferred embodiment, the process additionally comprises an enrichment of trans-1,3-diaminocyclohexane by extractive distillation in the presence of an extractant. The enrichment by extractive distillation is preferably carried out before the reaction according to the invention with carbon dioxide or a reactive carbonic acid derivative. That is to say, a trans-enriched 1,3-diaminocyclohexane mixture obtained in the extractive distillation is reacted according to the invention with carbon dioxide or a reactive carbonic acid derivative, as a result of which further trans enrichment occurs and substantially pure trans-1,3-diaminocyclohexane can finally be obtained.

The 1,3-diaminocyclohexane starting mixture for the extractive distillation preferably has a proportion of trans isomers of from 5 to 60% by weight and a proportion of cis isomers of from 40 to 95% by weight, particularly preferably a proportion of trans isomers of from 10 to 55% by weight and a proportion of cis isomers of from 45 to 90% by weight and very particularly preferably a proportion of trans isomers of from 20 to 50% by weight and a proportion of cis isomers of from 50 to 80% by weight, in each case based on the total amount of 1,3-diaminocyclohexane comprised in the starting mixture. In the trans-enriched 1,3-diaminocyclohexane mixture, the proportion of trans isomers, based on the total amount of 1,3-diaminocyclohexane comprised in the mixture, is greater than the proportion of trans isomers in the starting mixture. The trans-enriched 1,3-diaminocyclohexane mixture preferably has a proportion of trans isomers of more than 60% by weight and a proportion of cis isomers of less than 40% by weight, particularly preferably a proportion of trans isomers of from 60 to 80% by weight and a proportion of cis isomers of from 40 to 20% by weight and very particularly preferably a proportion of trans isomers of from 90 to 99.99% by weight and a proportion of cis isomers of from 10 to 0.01% by weight, in each case based on the total amount of 1,3-diaminocyclohexane comprised in the trans-enriched 1,3diaminocyclohexane mixture.

Possible extractants are compounds which form a high-boiling azeotrope with the cis isomers of the 1,3-diaminocyclohexane. The extractant appropriately has a boiling point which is at least 5° C. above the boiling point of the lowest-boiling 1,3-diaminocyclohexane isomer in the starting mixture. The extractant is present in liquid form at the temperature at the bottom of the extractive distillation. It comprises at least two functional groups selected from among hydroxyl and amino groups in the molecule. Suitable extractants are selected from among polyols, amino alcohols and polyamines.

Suitable polyols include ethylene glycol, 1,2-propanediol, 2-methylpropane-1,3-diol, 1,2-butanediol, 2,3-butanediol, 2-methylbutane-1,2-diol, 3-methylbutane-1,2-diol, 3-methyl-1,3-butanediol, 1,2-pentanediol, 1,3-pentanediol, 2,4-pentanediol, 2,3-pentanediol, 1,2-hexanediol, cis-1,2-cyclopentanediol, trans-1,2-cyclopentanediol, cis-1,2-cyclohexanediol, trans-1,2-cyclohexanediol, 1,3-propanediol, 2-methyl-1,3-propanediol, 2,2-dimethyl-1,3-propanediol (neopentyl glycol), 1,3-butanediol, 1,2-pentanediol, 2,4-pentanediol, 1,5-pentanediol, 1,3-hexanediol, 2,4-hexanediol, 1,3-cyclobutanediol, 1,3-cyclopentanediol, 1,3-cyclohexanediol, cis- and trans-1,4-butenediol, 1,4-butanediol, 2,3-dimethyl-1,4-butanediol, 2,2-dimethyl-1,4-butanediol, 1,4-pentanediol, 2,3-dimethyl-1,5-pentanediol, 1,4-hexanediol, 1,4-cyclohexanediol, 1,3,6-hexanetriol, 1,2,3-hexanetriol, 1,2,6-hexanetriol, glycerol, diglycerol, sorbitol, pentaerythritol, diethylene glycol, triethylene glycol, dipropylene glycol.

Suitable amino alcohols include diethanolamine, N-methyldiethanolamine, N-propyldiethanolamine, N-butyldiethanolamine, triethanolamine, N-ethylpropanolamine, N-propylethanolamine, N,N-dipropylethanolamine, N-butylethanolamine, N,N-dibutylethanolamine, propanolamine, dipropanolamine, N-methyldipropanolamine, N-propyldipropanolamine, N-butyldipropanolamine, tripropanolamine, diisopropanolamine, N-methyldiisopropanolamine, triisopropanolamine, N-2-methylaminopropanol, 4-amino-1-butanol, 4-(2-hydroxyethyl)morpholine, pentanolamine, hydroxyethylpiperazine, N-(2-hydroxyethyl)aniline, N,N-di(2-hydroxyethyl)aniline, 3-amino-1-propanol.

Suitable polyamines include 2-(diisopropylamino)ethylamine, 3-(cyclohexylamino)-propylamine, dipropylenetriamine, triethylenetetramine, pentamethyldiethylenetriamine, 3-(2-aminoethylamino)propylamine, diethylenetriamine, isophoronediamine.

Very particular preference is given to glycerol, 1,3-propanediol, 1,4-butanediol, cis-1,4-butenediol, triethylene glycol, diglycerol, 1,5-pentanediol, 4-(2-hydroxyethyl)morpholine, N-(2-hydroxyethyl)aniline, triethanolamine, N-methyldiethanolamine. Very particular preference is given to, in particular, 1,3-propanediol and 1,4-butanediol.

In the extractive distillation, trans-enriched 1,3-diaminocyclohexane can be obtained overhead. The starting mixture and the extractant are particularly preferably fed separately from one another into a distillation column. The introduction of the extractant very particularly preferably occurs above the introduction of the starting mixture. The introduction of the extractant and of the starting mixture can be carried out in either liquid, gaseous or boiling liquid form.

trans-Enriched 1,3-diaminocyclohexane is usually taken off as distillate at the top of the column or as side stream. If the trans-enriched 1,3-diaminocyclohexane is taken off as a sidestream, the side offtake is preferably located at least 1 theoretical plate, particularly preferably at least 5 theoretical plates, above the point of introduction of the extractant.

The proportion of extractant in the trans-enriched 1,3-diaminocyclohexane is usually small when a column having a high separating power is used and there are a sufficient number of separation stages between the introduction of the extractant and the outlet for the trans-enriched 1,3-diaminocyclohexane (in the distillate and/or the side offtake stream). A further possibility for reducing the proportion of extractant in the trans-enriched 1,3-diaminocyclohexane is to carry out a second distillation step.

The invention is illustrated by the following examples:

The following abbreviations are used:

MDACH: 4-Methylcyclohexane-1,3-diamine

### Example 1: Conversion of cis-MDACH Into Urea in the Presence of CO₂

Aqueous mixtures comprising 10% by weight of MDACH (60% cis isomer/40% trans isomer) were made up. In each case 8 ml of a mixture which was not loaded and a mixture loaded with 15 standard m³/t of CO₂ were made inert by means of nitrogen and introduced into 10 ml stainless steel (1.4571) autoclaves. The autoclaves were subsequently closed, heated to 160° C. in an oil bath and maintained there at 160° C. for 5 days. After cooling of the autoclaves, the latter were opened and sampled. For this purpose, the residual CO₂ was firstly stripped from the solution by means of N₂ and the solutions which were virtually free of acidic gas were subsequently analyzed in a gas chromatograph to determine the amine content and the secondary components formed. The composition of the samples is summarized in table 1.

TABLE 1
Composition of 10% MDACH solutions
(based on GC areas, without water).

| Sample designation | A | B | C |
|---|---|---|---|
| Total MDACH cis isomers | 61.65 | 61.47 | 34.18 |
| Total MDACH trans isomers | 38.35 | 38.53 | 38.17 |
| Urea | 0 | 0 | 27.65 |

A: untreated, B: 125 h at 160° C. without CO₂, C: 125 h at 160° C. with CO₂.

### Example 2: Comparison of Various MDACH Concentrations at Different CO₂ Loadings

trans-enriched MDACH (20% cis isomer/80% trans isomer) was obtained by extractive distillation using 1,3-propylene glycol as extractant. Aqueous mixtures comprising 10, 30 and 50% by weight of MDACH (20% cis isomer/80% trans isomer) were made up. In each case 8 ml of solution having different loadings (for details, see table 2) were made inert by means of nitrogen and introduced into 10 ml stainless steel (1.4571) autoclaves. The autoclaves were subsequently closed, heated to 160° C. in an oil bath and maintained there at 160° C. for 5 days. After cooling of the autoclaves, these were opened and sampled. For this purpose, the residual CO₂ was firstly stripped from the solutions by means of N₂ and the solutions which were virtually free of acidic gas were subsequently analyzed in a gas chromatograph to determine the amine content and the secondary components formed. The composition of the samples is summarized in table 2.

TABLE 2
Composition of various MDACH solutions (based
on GC areas, without water), with different initial
MDACH concentrations and CO₂ loadings.

| | A | B | C | D | C |
|---|---|---|---|---|---|
| Content of MDACH in | 10 | 10 | 10 | 30 | 50 |
| water [% by weight] | | | | | |
| 125 h at 160° C. | no | yes | yes | yes | yes |
| CO₂ loading [standard | 0 | 15 | 27 | 69 | 100 |
| m³/t] | | | | | |
| Total MDACH cis | 19.23 | 4.92 | 1.23 | 1.51 | 0.11 |
| isomers | | | | | |
| Total MDACH trans | 80.61 | 79.55 | 79.06 | 71.81 | 64.63 |
| isomers | | | | | |
| Urea | 0 | 15.53 | 19.71 | 26.68 | 35.26 |
| trans isomer based on | 80.7% | 94.2% | 98.4% | 97.9% | 99.8% |
| cis- and trans-MDACH | | | | | |

### Example 3: Comparison of Various Reaction Times

Aqueous mixtures comprising 10% by weight of MDACH (60% cis isomer/40% trans isomer) were made up. In each case 8 ml of fully loaded solution (27 standard m³/t) were made inert by means of nitrogen and introduced into 10 ml stainless steel (1.4571) autoclaves. The autoclaves were subsequently closed, heated to 160° C. in an oil bath and maintained there at 160° C. for 2, 4 and 6 days. After cooling the autoclaves, these were opened and sampled. For this purpose, the residual CO₂ was firstly stripped from the solutions by means of N₂ and the solutions which were virtually free of acidic gas were subsequently analyzed in a gas chromatograph to determine the amine content and the secondary components formed. The composition of the samples is summarized in table 3.

TABLE 3
Composition of MDACH solutions (based on GC areas, without
water) after different times of the experiment, T = 160°
C., initial CO₂ loading: 27 standard m³/t.

| | A | B | C | D |
|---|---|---|---|---|
| Duration of treatment with CO₂ gas [h] | 0 | 48 | 96 | 144 |
| Total MDACH cis isomers | 62.13 | 40.60 | 26.42 | 16.80 |
| Total MDACH trans isomers | 37.87 | 38.15 | 38.11 | 36.93 |
| Urea | 0 | 21.25 | 35.46 | 46.27 |

### Example 4

In a 160 ml autoclave made of stainless steel with an inclined blade stirrer, 12 g of MDACH were dissolved in 28 g of water. The autoclave was closed and brought to 25° C. While stirring at 500 revolutions per minute, 60 bar of CO₂ were injected and the pressure was kept constant by introducing further CO₂ when the pressure decreased. After stirring for one hour, the pressure was brought down to 2 bar. The temperature was subsequently increased to 180° C. while stirring at 200 rpm, and after this temperature had been reached, stirring was continued for 20 hours at 500 rpm. After the reaction time had elapsed, the autoclave was cooled to room temperature and vented. The partially solid contents were transferred to a flask. This procedure was repeated twice, and the combined reaction outputs from the three experiments were filtered on a suction filter and the filter cake was washed with 2×20 ml of ice water. After drying of the resulting moist filter cake under reduced pressure for 17 hours, 16.3 g of urea were obtained. The content of urea according to GC analysis was 98.1%; the remainder consisted of MDACH.

### Example 5

In a 160 ml autoclave made of stainless steel with an inclined blade stirrer, 12 g of MDACH were dissolved in 28 g of water. The autoclave was closed and brought to 25° C. While stirring at 500 revolutions per minute, 60 bar of CO₂ were injected and the pressure was kept constant by introducing further CO₂ when the pressure decreased. After stirring for one hour, the pressure was brought down to 2 bar. The temperature was subsequently increased to 160° C. while stirring at 200 rpm, and after this temperature had been reached, stirring was continued for 14 hours at 500 rpm. After this time, a sample was analyzed by GC. The cis/trans ratio was 4.1:95.9. The contents of the autoclave were treated once more as described above and again heated for 14 hours. The ratio of cis to trans isomer was then 0.2:99.8. The mixture of products consisted of 80% of MCDA, 15.3% of cis-urea and 4.7% of other products whose identity was not elucidated.

1. A process for discriminating cis- and trans-1,3diaminocyclohexanes, the process comprising reacting a mixture of cis- and trans-1,3-diaminocyclohexanes with carbon dioxide or a reactive carbonic acid derivative and selectively obtaining the urea of cis-1,3-diaminocyclohexane.

2. The process of claim 1, wherein the reactive carbonic acid derivative is selected from among urea, carbonic esters and phosgene.

3. The process of claim 1, further comprising separating the urea of cis-1,3-diaminocyclohexane.

4. The process of claim 3, wherein the separating comprises a mechanical separation method.

5. The process of claim 1, wherein the reacting is carried out in an aqueous solution.

6. The process of claim 5, wherein an aqueous solution of cis- and trans-1,3-diaminocyclohexanes comprising carbon dioxide is heated under autogenous pressure in a pressure vessel.

7. The process of claim 1, additionally comprising an enrichment of trans-1,3-diaminocyclohexane by extractive distillation in the presence of an extractant.

8. The process of claim 7, wherein the extractant is selected from among polyols, amino alcohols and polyamines.

9. The process of claim 1, wherein the 1,3-diaminocyclohexane comprises at least one C₁-C₆-alkyl group in an α position relative to at least one amino group.

10. The process of claim 9, wherein the 1,3-diaminocyclohexane is 4-methyl cyclohexane-1,3-diamine, 2-methylcyclohexane-1,3-diamine or a mixture thereof.

11. A 4-Methylcyclohexane-1,3-diamine, 2-methylcyclohexane-1,3-diamine or a mixture thereof, having a trans content of 99 mol % or more based on the sum of 4-methylcyclohexane-1,3-diamine and 2-methylcyclohexane-1,3-diamine.
